(19) United States
(12) Patent Application Publication
(10) Pub. No.: US 2009/0047231 A1
(43) Pub. Date: Feb. 19, 2009

(54) AQUEOUS HAIR COSMETIC COMPOSITION

(75) Inventors: Fumiko Sazanami, Tokyo (JP); Hiroto Tanamachi, Tokyo (JP); Minoru Nagai, Tochigi (JP); Mio Ishita, Tochigi (JP)

Correspondence Address: OBLON, SPIVAK, MCCLELLAND MAIER & NEUSTADT, P.C., 1940 DUKE STREET, ALEXANDRIA, VA, 22314, US

(73) Assignee: KAO CORPORATION, Tokyo (JP)

(21) Appl. No.: 12/088,349

(22) PCT Filed: Nov. 30, 2006
(86) PCT No.: PCT/JP2006/324411

§ 371 Date: Mar. 27, 2008

(30) Foreign Application Priority Data
Dec. 1, 2005 (JP) 2005-347482
Dec. 1, 2005 (JP) 2005-347483
Aug. 9, 2006 (JP) 2006-216965

(51) Int. Cl. A61K 8/41; A61Q 5/00

(52) U.S. Cl. 424/70.27

(57) ABSTRACT

Provided is an aqueous hair cosmetic composition containing (A) a tertiary amine type cationic surfactant, (B) an aromatic alcohol, (C) a branched fatty acid or salt thereof, and water, wherein Component (B) and Component (C) at a (B)/(C) weight ratio falls within a range of from 100/1 to 1/10. The aqueous hair cosmetic composition of the present invention can repair or prevent the hair damage and fatigue breakdown and give good flexibility and a supple touch to the hair during from wetting to even after drying.

### FIELD OF THE INVENTION

The present invention relates to an aqueous hair cosmetic composition containing a tertiary amine type cationic surfactant.

### BACKGROUND OF THE INVENTION

Hair tends to be excessively dry because it is always exposed to sunlight and cannot avoid the influence of ultraviolet rays, heat and drying. Daily shampooing, brushing and blow drying also have an adverse influence. It has been elucidated that hair damage such as loss of luster or excessive drying occurs because the hair becomes porous by the heat from a drier. In recent years, it has been common to enjoy changing the appearance of hair freely such as changing hair color (coloring) and changing hair style (permanent waving) so that coloring and permanent waving have been carried out with increased frequency. The hair subjected to coloring treatment or permanent waving treatment is said to become porous owing to the chemical factors of a hair dye or permanent waving agent used for the treatment. In addition, daily hair care routine (shampooing, finger combing, and brushing) continues to impose small strain to the hair, whereby cracks, voids, cleavages or cuts grow inside the hair.

Various attempts have therefore been made to repair hair damage. For the purpose of repairing or preventing hair damage and providing hair with an excellent feeling, there are proposed, for example, a hair cosmetic composition using an ether type tertiary amine, a higher alcohol and an organic solvent in combination (JP-A-2004-67534), a hair cosmetic composition using an amidoamine compound or salt thereof, a higher alcohol and an organic solvent in combination (JP-A-2003-81780), a hair care composition containing a hydroxy ether amine compound, an acid, a higher alcohol and an oil component (JP-A-2004-323495), and a hair cosmetic composition containing a branched fatty acid (JP-A-4-173719).

These hair cosmetic compositions cannot however satisfactorily repair or prevent fatigue breakage (split ends, breakage) due to hair coloring or the like.

### SUMMARY OF THE INVENTION

In the present invention, there is thus provided an aqueous hair cosmetic composition containing components (A), (B) and (C), and water.

(A) A tertiary amine type cationic surfactant
(B) An aromatic alcohol represented by the formula (2):

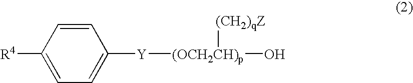

(wherein, R⁴ represents a hydrogen atom, a methyl group or a methoxy group, Y represents a single bond or a linear or branched C₁₋₃ alkylene or alkenylene group, Z represents a hydrogen atom or a hydroxyl group and p and q each stands for a number from 0 to 5).
(C) A branched fatty acid represented by the following formula (3) or salt thereof:

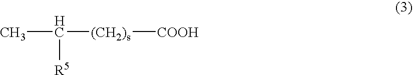

(wherein, R⁵ represents a methyl or ethyl group and s stands for an integer of from 3 to 36).

### DETAIL DESCRIPTION OF THE INVENTION

The present invention relates to an aqueous hair cosmetic composition capable of repairing or preventing hair damage and fatigue breakage due to chemical treatment, blow drying or daily hair care routine and at the same time capable of giving good flexibility and a supple touch to the hair from wetting to even after drying.

The present inventors have found that an aqueous hair cosmetic composition satisfying the above-described demand is available by using a tertiary amine type cationic surfactant, an aromatic alcohol and a branched fatty acid in combination.

Examples of the tertiary amine type cationic surfactant as Component (A) include ether amines represented by the formula (1-1), amidoamines represented by the formula (1-2), hydroxy ether amines represented by the formula (1-3), and alkyldimethylamines represented by the formula (1-4), and salts thereof.

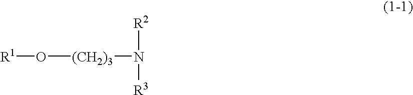

(wherein, R¹ represents a linear or branched C₆₋₂₄ alkyl or alkenyl group, R² and R³ may be the same or different and each represents a C₁₋₆ alkyl group or a group -(AO)ₘH (in which A represents a C₂₋₄ alkylene group, and m stands for a number from 1 to 6 with the proviso that m pieces of A may be the same or different and they may be arranged in any order)).

R—CONH—(CH₂)ᵣ—N(R′)₂ (1-2)

(wherein, R represents an aliphatic C₁₋₂₃ hydrocarbon group, R's may be the same or different and each represents a hydrogen atom or a C₁₋₄ alkyl group, and r stands for a number from 2 to 4).

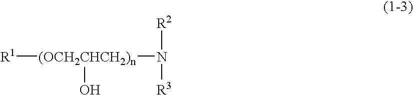

(wherein, R¹, R² and R³ have the same meanings as described above and n stands for a number from 1 to 5).

R¹—N(CH₃)₂ (1-4)

(wherein, R¹ has the same meaning as described above).

In the ether amine represented by the formula (1-1), R¹ is a linear or branched C₆₋₂₄ alkyl or alkenyl group. A linear or branched C₁₂₋₂₄ alkyl or alkenyl group, specifically C₁₄₋₂₂ alkyl or alkenyl group is preferred because it can give good flexibility and lubrication to the hair from wetting to even after drying. In particular, lubrication after drying is excellent. Of these groups, the alkyl groups are preferred.

R² and R³ each independently represents a C₁₋₆ alkyl group or a group -(AO)ₘH (in which A and m have the same meanings as described above). A C₁₋₆ alkyl group or a group —(CH₂CH₂O)ₘH (in which m stands for a number from 1 to 3, preferably 1) is preferred because it can give good flexibility and lubrication to the hair during from wetting to even after drying. In particular, lubrication after drying is excellent. It is more preferred that at least one of R² and R³ represents a C₁₋₆ alkyl group, still more preferably a methyl or ethyl group. It is preferred that R² and R³ represent the same group.

Specific preferred examples of the ether amine (1-1) include N,N-dimethyl-3-hexadecyloxypropylamine and N,N-dimethyl-3-octadecyloxypropylamine.

In the amidoamine compound represented by the formula (1-2), examples of the fatty acid residue (acyl group) represented by RCO in the formula (1-2) include lauroyl, myristoyl, palmitoyl, stearoyl, oleoyl and behenoyl groups. The fatty acid residues represented by RCO are preferably composed of 60 wt. % or greater of fatty acid residues having at least 20 carbon atoms, 3 wt. % or greater of a fatty acid residue having 20 carbon atoms, and from 50 to 95 wt. % of a fatty acid residue having 22 carbon atoms, each in all the fatty acid residues, from the standpoint of improving flexibility and lubrication during from wetting to even after drying. When priority is given to the lubrication after drying, the fatty acid residues are composed of 75 wt. % or greater, more preferably 90 wt. % or greater of fatty acid residues having at least 20 carbon atoms, 4 wt. % or greater, more preferably 5 wt. % or greater of a fatty acid residue having 20 carbon atoms, and from 55 to 95 wt. %, more preferably from 70 to 95 wt. %, still more preferably from 80 to 95 wt. % of a fatty acid residue having 22 carbon atoms. As R′, a methyl group, ethyl group, and propyl group are preferred, with a methyl group being more preferred. As r, 2 or 3 is preferred.

Specific examples of the amidoamine compound (1-2) include stearic acid dimethylaminoethylamide, stearic acid dimethylaminopropylamide, stearic acid diethylaminoethylamide, stearic acid diethylaminopropylamide, stearic acid dipropylaminoethylamide, stearic acid dipropylaminopropylamide, palmitic acid dimethylaminoethylamide, palmitic acid dimethylaminopropylamide, myristic acid dimethylaminoethylamide, myristic acid dimethylaminopropylamide, behenic acid dimethylaminoethylamide, behenic acid dimethylaminopropylamide, arachidic acid dimethylaminoethylamide and arachidic acid dimethylaminopropylamide. Of these, stearic acid diethylaminoethylamide and stearic acid dimethylaminopropylamide are preferred from the standpoints of performance, stability and easy availability.

In the hydroxyetheramine represented by the formula (1-3), examples of R¹, R² and R³ are preferably similar to those in the formula (1-1) and n is preferably 1.

Specific preferred examples of the hydroxyetheramine (1-3) include hexadecyloxy(2-hydroxypropyl)dimethylamine, octadecyloxy(2-hydroxypropyl)dimethylamine and behenyloxy(2-hydroxypropyl)dimethylamine.

It is preferred that a portion or whole of each of the tertiary amine compounds (1-1) to (1-3) to be used in the present invention has been neutralized with an inorganic acid or an organic acid.

Examples of the inorganic acid include hydrochloric acid, sulfuric acid and phosphoric acid. Examples of the organic acid include monocarboxylic acids such as acetic acid and propionic acid, dicarboxylic acids such as malonic acid, succinic acid, glutaric acid, adipic acid, maleic acid, fumaric acid and phthalic acid, polycarboxylic acids such as polyglutamic acid, hydroxycarboxylic acids such as glycolic acid, lactic acid, hydroxyacrylic acid, glyceric acid, malic acid, tartaric acid and citric acid, and acidic amino acids such as glutamic acid and aspartic acid. Of these, inorganic acids, dicarboxylic acids, hydroxycarboxylic acids and acidic amino acids are preferred. As the inorganic acid, hydrochloric acid is preferred. As the dicarboxylic acid, maleic acid and succinic acid are preferred. As the hydroxycarboxylic acid, glycolic acid, lactic acid, and malic acid are preferred. As the acidic amino acid, glutamic acid is preferred.

As the organic acid, the branched fatty acid as Component (C) may be employed. Alternatively, the amine compound (1-1) to (1-3) may be mixed and neutralized with the branched fatty acid in advance and the mixture may be used as an acid addition salt. In the aqueous hair cosmetic composition of the present invention, Component (A) and Component (C) are presumed to form a hydrophobic composite.

The inorganic acid and/or organic acid are added preferably in an amount of from 0.1 to 10 moles, more preferably from 0.3 to 4 moles per mole of the amine compound (1-1) to (1-3) in order to effectively reduce an amine odor and improve conditioning effects such as flexibility and lubrication.

As Component (A), two or more tertiary amine type cationic surfactants may be used in combination. When Component (A) is the etheramine (1-1) or hydroxyetheramine (1-3), its content, in terms of amine, is preferably from 0.1 to 20 wt. %, more preferably from 0.3 to 15 wt. %, still more preferably from 0.5 to 10 wt. % in order to give good flexibility and lubrication during application of the resulting composition. When it is the amidoamine (1-2), its content, in terms of amine, is preferably from 0.01 to 20 wt. %, more preferably from 0.05 to 15 wt. %, still more preferably from 0.1 to 10 wt. %.

Examples of the aromatic alcohol as Component (B) include benzyl alcohol, cinnamyl alcohol, phenethyl alcohol, p-anisyl alcohol, p-methylbenzyl alcohol, phenoxyethanol, and 2-benzyloxyethanol. Of these, benzyl alcohol and 2-benzyloxyethanol are preferred.

As Component (B), two or more aromatic alcohols may be used in combination. Its content in the aqueous hair cosmetic composition of the present invention is preferably from 0.05 to 30 wt. %, more preferably from 0.1 to 10 wt. % in order to give good flexibility and a supple touch to the hair during application of the resulting hair cosmetic composition.

The branched fatty acid as Component (C) can be separated or extracted from the hair or the like in accordance with, for example, the method described in LIPIDS, Vol. 23, No. 9, p 878-881 (1988). Alternatively, it can be synthesized in accordance with the method described in Patent Document No. 2, that is, WO98/30532. The branched fatty acid is represented by the formula (3) and has preferably from 7 to 40 carbon atoms, more preferably from 8 to 40 carbon atoms, even more preferably from 10 to 22 carbon atoms, each in total. Specific examples include 18-methyleicosanoic acid, 14-methylpentadecanoic acid, 14-methylhexadecanoic acid, 16-methylheptadecanoic acid and 16-methyloctadecanoic acid. Examples of the salts of these branched fatty acids include alkali metal salts such as sodium salts, lithium salts and potassium salts; alkaline earth metal salts such as calcium salts and magnesium salts, ammonium salts, organic amine salts such as triethanolamine salts, diethanolamine salts and monoethanolamine salts, and basic amino acid salts such as lysine salts and arginine salts.

Examples of the branched fatty acid obtained by extraction include lanolin fatty acids obtained by extraction of lanolin. The lanolin fatty acids each contains about 50 wt. % of a methyl-branched long-chain fatty acid which is called iso fatty acid or anteiso fatty acid. Specific examples include “Crodacid 18-MEA” (Croda Japan K.K.), “Skliro” (Croda Japan K.K.) and “FA-NH” (Nippon Fine Chemical).

As Component (C), two or more branched fatty acids or salts thereof may be used in combination. A mixture of a synthesized branched fatty acid and an extracted one may be used. The content of Component (C) in the aqueous hair cosmetic composition of the present invention is preferably from 0.01 to 10 wt. %, more preferably from 0.05 to 5 wt. % in view of its effect in recovering from or preventing hair damage.

A Component (B)/Component (C) weight ratio preferably falls within a range of from 100/1 to 1/10, more preferably from 10/1 to 1/10, still more preferably from 5/1 to 1/5 in order not only to produce an effect of repairing and preventing hair damage or fatigue breakage and giving flexibility and suppleness to the hair but also to ensure good stability and feeling upon use.

The amount of water contained in the aqueous hair cosmetic composition of the present invention is preferably from 50 to 95 wt. %, more preferably from 60 to 90 wt. %.

The aqueous hair cosmetic composition of the present invention may further contain an amphipathic amide lipid as Component (D) in order to improve the external repairing effect of the hair. Examples of the amphipathic amide lipid include diamide compounds represented by the formula (4) and ceramides represented by the formula (5).

(i) Diamide compounds represented by the formula (4):

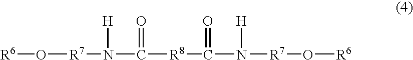

(wherein, R⁶ represents a linear or branched C₁₋₁₂ hydrocarbon group which may be substituted with a hydroxy group(s) and/or alkoxy group(s), R⁷ represents a linear or branched divalent C₁₋₅ hydrocarbon group and R⁸ represents a linear or branched divalent C₁₋₂₂ hydrocarbon group).

As R⁶ in formula (4), linear or branched C₁₋₁₂ alkyl groups which may be substituted with from 1 to 3 groups selected from a hydroxy group and C₁₋₆ alkoxy groups are preferred. Of these, unsubstituted C₁₋₁₂ alkyl groups and C₂₋₁₂ alkyl groups substituted with 1 or 2 hydroxy groups and one C₁₋₆ alkoxy group or with one hydroxy group and one C₁₋₆ alkoxy group are more preferred. Specific examples include methyl, ethyl, propyl, butyl, hexyl, dodecyl, 2-methylpropyl, 2-ethylhexyl, 2-hydroxyethyl, 9-hydroxynonyl, 2,3-dihydroxypropyl, 2-methoxyethyl, 2-hydroxy-3-methoxypropyl and 9-methoxynonyl groups, of which 2-hydroxyethyl, methyl, dodecyl and 2-methoxyethyl groups are preferred.

As R⁷ in formula (4), linear or branched C₂₋₅ alkylene groups are preferred, and linear or branched C₂₋₃ alkylene groups are more preferred. Specific examples include ethylene, trimethylene, tetramethylene, pentamethylene, 1-methylethylene, 2-methylethylene, 1-methyltrimethylene, 2-methyltrimethylene, 1,1-dimethylethylene and 2-ethyltrimethylene groups. Of these, ethylene and trimethylene groups are preferred.

As R⁸ in formula (4), linear or branched divalent C₂₋₂₂ hydrocarbon groups are preferred, and linear or branched C₁₁₋₂₂ alkylene groups and alkenylene groups having from 1 to 4 double bonds are more preferred. Specific examples include ethylene, trimethylene, tetramethylene, hexamethylene, heptamethylene, octamethylene, decamethylene, undecamethylene, dodecamethylene, tridecamethylene, tetradecamethylene, hexadecamethylene, octadecamethylene, 1-methylethylene, 2-ethyltrimethylene, 1-methylheptamethylene, 2-methylheptamethylene, 1-butylhexamethylene, 2-methyl-5-ethylheptamethylene, 2,3,6-trimethylheptamethylene, 6-ethyldecamethylene, 7-methyltetradecamethylene, 7-ethylhexadecamethylene, 7,12-dimethyloctadecamethylene, 8,11-dimethyloctadecamethylene, 7,10-dimethyl-7-ethylhexadecamethylene, 1-octadecylethylene, ethenylene, 1-octadecenylethylene, 7,11-octadecadienylene, 7-ethenyl-9-hexadecamethylene, 7,12-dimethyl-7,11-octadecadienylene and 8,11-dimethyl-7,11-octadecadienylene groups. Of these, 7,12-dimethyloctadecamethylene, 7,12-dimethyl-7,11-octadecadienylene, octadecamethylene, undecamethylene and tridecamethylene groups are preferred.

Preferred diamide compounds (4) are compounds having the above-described preferred groups as R⁶, R⁷ and R⁸, respectively, in combination. Specific examples are the following compounds:

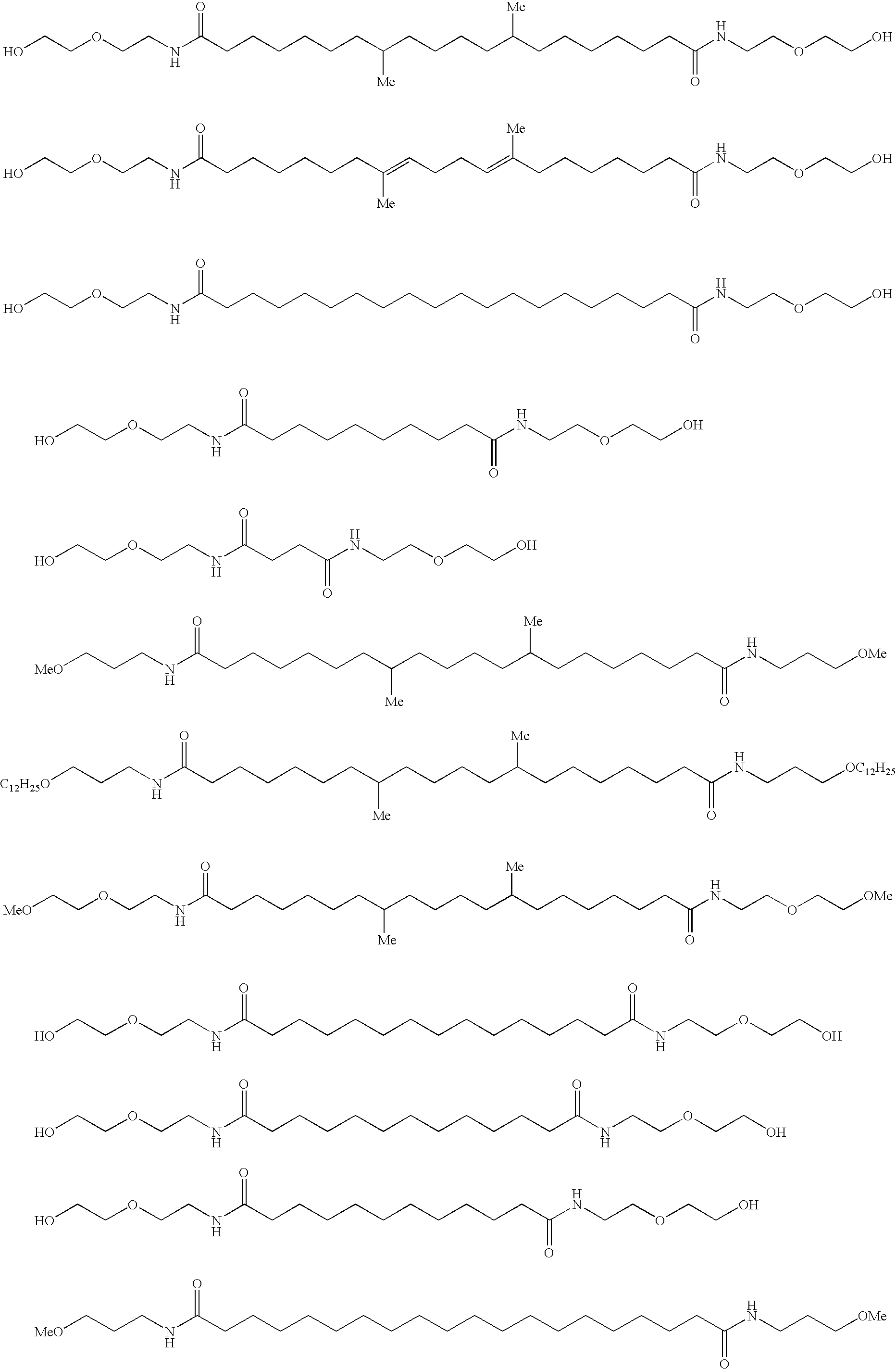

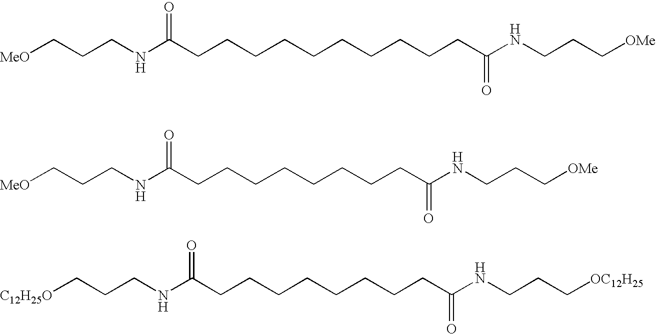

(ii) Ceramides represented by the following formula (5):

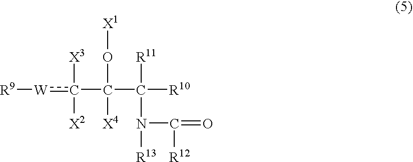

(wherein, R⁹ represents a linear, branched or cyclic, saturated or unsaturated C₄₋₃₀ hydrocarbon group which may be substituted with hydroxy, oxo or amino group(s), W represents a methylene group, a methine group or an oxygen atom, a broken line represents the presence or absence of a π bond, X¹ represents a hydrogen atom, an acetyl group or a glyceryl group, or forms an oxo group together with the adjacent oxygen atom, X², X³ and X⁴ each independently represent a hydrogen atom, a hydroxy group or an acetoxy group (with the proviso that when W represents a methine group, either X² or X³ represents a hydrogen atom and the other does not exist, and when —O—X¹ represents an oxo group, X⁴ does not exist), R¹⁰ and R¹¹ each independently represents a hydrogen atom, a hydroxy group, a hydroxymethyl group or an acetoxymethyl group, R¹² represents a linear, branched or cyclic, saturated C₅₋₃₅ hydrocarbon group which may be substituted with a hydroxy or amino group, or the saturated C₅₋₃₅ hydrocarbon group in which a linear, branched or cyclic, saturated or unsaturated C₈₋₂₂ fatty acid which may be substituted with hydroxy group(s) has been ester-bonded to the ω-position of the hydrocarbon group, and R¹³ represents a hydrogen atom or a linear or branched, saturated or unsaturated hydrocarbon group which may have substituent(s) selected from a hydroxy group, hydroxyalkoxy groups, alkoxy groups and an acetoxy group, and has 1 to 8 carbon atoms in total.

As R⁹ in formula (5), linear, branched or cyclic, saturated or unsaturated C₇₋₂₂ hydrocarbon groups which may be substituted with hydroxy group(s) are preferred. As X¹, a hydrogen atom and a glyceryl group are preferred. It is preferred that at most one of X², X³, and X⁴ represents a hydroxy group and the others represent a hydrogen atom. It is preferred that one of R¹⁰ and R¹¹ represents a hydrogen atom or a hydroxymethyl group and the other represents a hydrogen atom. In R¹², preferred examples of the fatty acid which may be ester-bonded or amide-bonded to the ω-position of the saturated hydrocarbon group include isostearic acid, 12-hydroxystearic acid and linoleic acid. As R¹³, a hydrogen atom and hydrocarbon groups which have 1 to 8 carbon atoms in total and may be substituted with 1 to 3 substituents selected from a hydroxy group, hydroxyalkoxy groups and alkoxy groups are preferred.

As ceramide (5), preferred are natural ceramides and natural type ceramides, and derivatives thereof represented by the below-described formulas (5a) (which will hereinafter be called “natural type ceramides (5a)”) and pseudo type ceramides represented by the formula (5b) (which will hereinafter be called “pseudo type ceramides (5b)).

(ii-1) Natural type ceramides (5a)

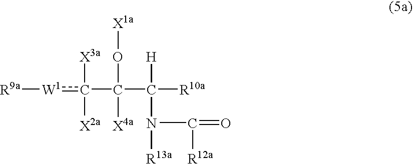

(wherein, R^{9a} represents a linear, branched or cyclic, saturated or unsaturated C₇₋₁₉ hydrocarbon group which may be substituted with a hydroxy group, W¹ represents a methylene or methine group, a broken line represents the presence or absence of a π bond, X^{1a} represents a hydrogen atom or, forms an oxo group together with the adjacent oxygen atom, X^{2a}, X^{3a} and X^{4a} each independently represent a hydrogen atom, a hydroxy group or an acetoxy group (with the proviso that when W¹ represents a methine group, one of X^{2a} and X^{3a} represents a hydrogen atom and the other does not exist, and when —O—X^{1a} represents an oxo group, X^{4a} does not exist), R^{10a} represents a hydroxymethyl group or an acetoxymethyl group, R^{12a} represents a linear, branched or cyclic, saturated C₅₋₃₀ hydrocarbon group which may be substituted with hydroxy group(s), or the saturated C₅₋₃₀ hydrocarbon group in which a linear or branched, saturated or unsaturated C₈₋₂₂ fatty acid which may be substituted with hydroxy group(s) has been ester-bonded to the ω-position of the hydrocarbon group, and R^{13a} represents a hydrogen atom or a C₁₋₄ alkyl group).

Preferred are compounds in which R^{9a} is a linear C₇₋₁₉, more preferably C₁₃₋₁₅, alkyl group; W¹ is a methine group and one of X^{2a} and X^{3a} is a hydrogen atom; and R^{12a} is a linear C₉₋₂₇ alkyl group which may be substituted with hydroxy group(s). In addition, X^{1a} preferably represents a hydrogen atom, or forms an oxo group together with an oxygen atom. Preferred examples of R^{12a} include a tricosyl group, a 1-hydroxypentadecyl group, a 1-hydroxytricosyl group, a heptadecyl group, a 1-hydroxyundecyl group and a nonacosyl group having a linoleic acid ester-bonded to the ω-position thereof.

Specific examples of the natural type ceramides include Ceramide Types 1 to 7 having the structures described below which are obtained by amidation of sphingosine, dihydrosphingosine, phytosphingosine or sphingadienine (for example, FIG. 2 of J. Lipid Res., 24: 759 (1983), and pig and human ceramides as described in FIG. 4 of J. Lipid Res., 35: 2069 (1994)) and N-alkyl derivatives (for example, N-methyl derivatives) thereof. Examples also include N-alkyl derivatives (e.g., N-methyl derivatives) of the above-described ceramides. They may be either a natural extract or synthesized product. Commercially available ones can also be used.

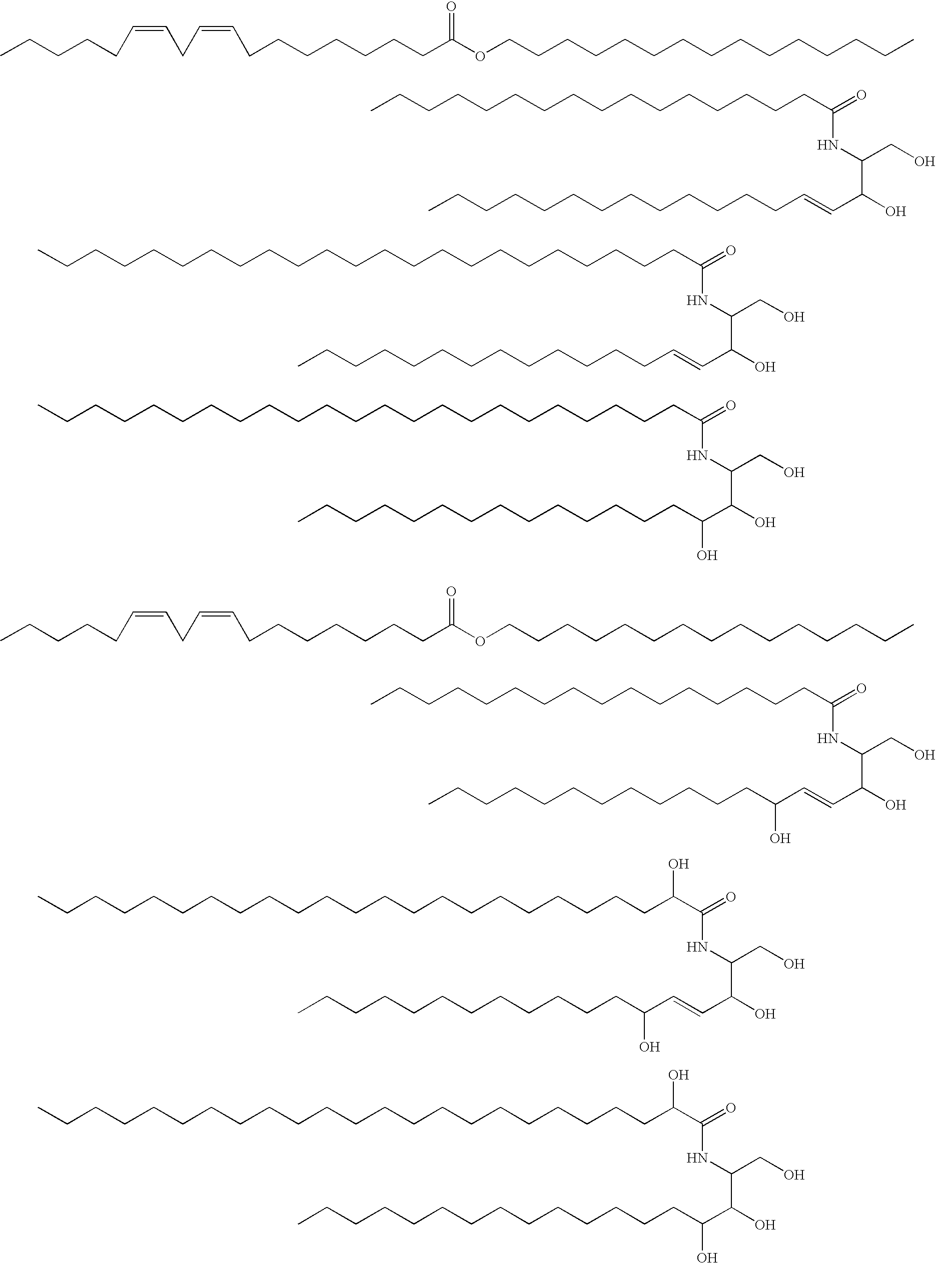

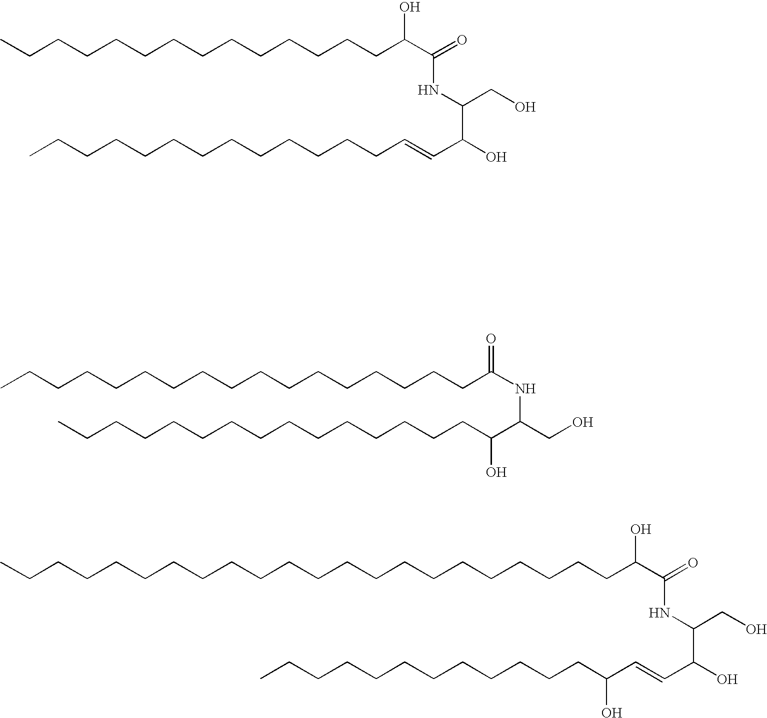

(ii-2) Pseudo type ceramides (5b)

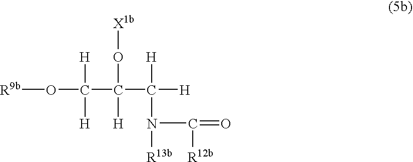

(wherein, R^{9b} represents a linear, branched or cyclic, saturated or unsaturated C₁₀₋₂₂ hydrocarbon group which may be substituted with hydroxy group(s), X^{1b} represents a hydrogen atom, an acetyl group or a glyceryl group, R^{12b} represents a linear, branched or cyclic, saturated or unsaturated C₅₋₂₂ hydrocarbon group which may be substituted with hydroxyl or amino group(s), or the hydrocarbon group in which a linear or branched, saturated or unsaturated C₈₋₂₂ fatty acid which may be substituted with hydroxy group(s) has been ester-bonded to the ω-position of the hydrocarbon group, and R^{13b} represents a hydrogen atom or an alkyl group which has from 1 to 8 carbon atoms in total and may be substituted with hydroxy, hydroxyalkoxy, alkoxy or acetoxy group(s).

Preferred as R^{12b} are a nonyl group, a tridecyl group, a pentadecyl group, an undecyl group having linoleic acid ester-bonded to the ω-position thereof, a pentadecyl group having linoleic acid ester-bonded to the ω-position thereof, a pentadecyl group having 12-hydroxystearic acid ester-bonded to the ω-position thereof, and an undecyl group having methyl-branched isostearic acid amide-bonded to the ω-position thereof. The hydroxyalkoxy or alkoxy groups for R^{13b} have preferably 1 to 8 carbon atoms.

As the pseudo type ceramides (5b), those having a hexadecyl group as R^{9b}, a hydrogen atom as X^{1b}, a pentadecyl group as R^{12b}, and a hydroxyethyl group as R^{13b}; those having a hexadecyl group as R^{9b}, a hydrogen atom as X^{1b}, a nonyl group as R^{12b}, and a hydroxyethyl group as R^{13b}; or those having a hexadecyl group as R^{9b}, a glyceryl group as X^{1b}, a tridecyl group as R^{12b}, and a 3-methoxypropyl group as R^{13b} are preferred, with those having a hexadecyl group as R^{9b}, a hydrogen atom as X^{1b}, a pentadecyl group as R^{12b}, and a hydroxyethyl group as R^{13b} being more preferred. Specific preferred examples include those represented by the following formulas:

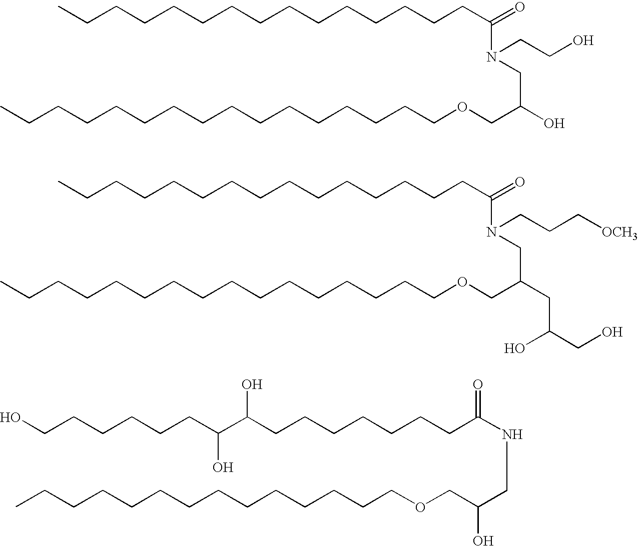

Two or more of these amphipathic amide lipids may be used in combination. Its (their) content in the aqueous hair cosmetic composition of the present invention is preferably from 0.01 to 10 wt. %, more preferably from 0.05 to 5 wt. % in view of recovering or preventing hair damage.

To the aqueous hair cosmetic composition of the present invention, a silicone, oil component, and quaternary ammonium salt type cationic surfactant and the like may be added in order to improve hair touch further. For example, silicones described below can be employed.

(i) Highly polymerized dimethylpolysiloxanes

Examples include “BY11-026” and “BY22-19” (each, product of Dow Corning Toray), and “FZ-3125” (product of Nippon Unicar).

The highly polymerized dimethylpolysiloxane dissolved or dispersed in a liquid oil (for example, the below-described (ii) dimethylpolysiloxane oil, (iii) liquid silicone oil such as cyclic silicone or liquid hydrocarbon oil such as isoparaffin) can also be used.

(ii) Dimethylpolysiloxane oil represented by the following formula:

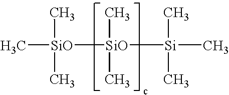

(wherein, c stands for an integer of from 0 to 650).

Specific examples include commercially available products such as “SH200C Series having a viscosity of 1 cs, 50 cs, 200 cs, 1000 cs and 5000 cs, respectively” (each product of Dow Corning Toray).

(iii) Cyclic silicone represented by the following formula:

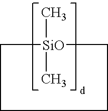

(wherein, d stands for an integer of from 3 to 7).

Specific examples include dodecamethylcyclohexasiloxane, octamethylcyclotetrasiloxane and decamethylcyclopentasiloxane. Examples of the commercially available product include “SH244” and “SH245” (each, product of Dow Corning Toray).

(iv) Amino-modified silicones represented by the following formula:

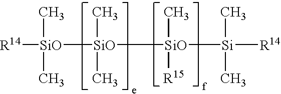

(wherein, R¹⁴ represent a similar group to that of R¹⁵ or a methyl or hydroxyl group, R¹⁵ represents a reactive functional group represented by —R¹⁶-Q (in which R¹⁶ represents a divalent C₃₋₆ hydrocarbon group and Q represents a group containing a primary, secondary or tertiary amino group or an ammonium-containing group), and e and f each stands for a positive integer and e+f varies depending on the molecular weight. Preferable average molecular weight is from 3000 to 100000).

Examples include “SS-3551”, “SF8452C”, “DC929” and “DC8500” (each, product of Down Corning Toray) and “KT 1989” (product of GE Toshiba). When the amino-modified silicone is used in the form of an aqueous emulsion, the amount of the amino-modified silicone contained in the aqueous emulsion is preferably from 20 to 60 wt. %, more preferably from 30 to 50 wt. %. Preferred examples of the aqueous emulsion of an amino-modified silicone include “SM8704C” (product of Dow Corning Toray).

(v) Other silicones

Examples of the silicone other than those described above include polyether modified silicones, methylphenylpolysiloxane, fatty acid modified silicones, alcohol modified silicones, alkoxy modified silicones, epoxy modified silicones, fluorine modified silicones, cyclic silicones, and alkyl modified silicones.

When the silicone compound is incorporated in the aqueous hair cosmetic composition of the present invention, its content therein is preferably from 0.1 to 15 wt. %, more preferably from 0.5 to 10 wt. %.

Examples of the oil component include higher fatty acids other than Component (C) such as capric acid, lauric acid, myristic acid, palmitic acid, stearic acid, behenic acid, oleic acid, and coconut fatty acid; and hydrocarbon oils such as liquid paraffin, liquid isoparaffin, vaseline, squalene and squalane. Examples further include natural oils such as camellia oil, Macadamia nut oil, corn oil, olive oil, avocado oil, castor oil, safflower oil, jojoba oil, sunflower oil, rapeseed oil, sesame oil, soybean oil and meadow foam oil; and ester oils such as isopropyl myristate, isopropyl palmitate, myristyl myristate, octyl palmitate, stearyl stearate, isocetyl stearate, isononyl isononanoate, isotridecyl isononanoate, stearic acid hydrogenated castor oil, hydroxystearic acid hydrogenated castor oil, glyceryl tri-2-ethylhexanoate, pentaerythritol tetra-2-ethylhexanoate, neopentyl glycol dicaprate, diglyceryl diisostearate and esters between dipentaerythritol and a mixed fatty acid such as hydroxystearic acid/stearic acid/rosic acid.

Examples of the quaternary ammonium salt type cationic surfactant include following compounds (i) to (iii).

(i) Alkyltrimethylammonium salts

Usable are, for example, compounds represented by the following formula:

R¹⁷—N⁺(CH₃)₃X⁻

(wherein, R¹⁷ represents a C₁₂₋₂₂ alkyl group and X⁻ represents a halide ion (chloride ion or bromide ion) or an alkyl sulfate ion having 1 or 2 carbon atoms).
(ii) Alkoxytrimethylammonium salts

Usable are, for example, compounds represented by the following formula:

R¹⁸—O—R¹⁹—N⁺(CH₃)₃X⁻

(wherein, R¹⁸ represents a C₁₂₋₂₂ alkyl group and R¹⁹ represents an ethylene or propylene group and X⁻ has the same meaning as described above).
(iii) Dialkyldimethylammonium salts

Usable are, for example, compounds represented by the following formula:

R²⁰₂—N⁺(CH₃)₂X⁻

(wherein, R²⁰ represents a C₁₂₋₂₂ alkyl or benzyl group and X⁻ has the same meaning as described above).

Examples of the quaternary ammonium salt type cationic surfactants other than those described above in (i) to (iii) include lanolin fatty acid aminopropylethyldimethylammonium ethyl sulfate (ethyl sulfate salt of alkanoyl aminopropyldimethylethylammonium, the alkanoyl group is derived from lanolin), lanolin fatty acid aminoethyltriethylammonium ethyl sulfate, lanolin fatty acid aminopropyltriethylammonium ethyl sulfate, lanolin fatty acid aminoethyltrimethylammonium methyl sulfate, lanolin fatty acid aminopropylethyldimethylammonium methyl sulfate, isoalkanoic acid (C₁₄-C₂₀) aminopropylethyldimethylammonium ethyl sulfate, isoalkanoic acid (C₁₈-C₂₂) aminopropylethyldimethylammonium ethyl sulfate, isostearic acid aminopropylethyldimethylammonium ethyl sulfate, isononanoic acid aminopropylethyldimethylammonium ethyl sulfate, and alkyltrimethylammonium saccharins.

As the cationic surfactant, two or more of the above-described ones may be used in combination. The content of the cationic surfactant(s) is preferably from 0.01 to 20 wt. %, more preferably from 0.1 to 15 wt. %, still more preferably from 0.5 to 10 wt. % in order to give good flexibility and lubrication to the hair during application of the resulting hair cosmetic composition.

The aqueous hair cosmetic composition of the present invention may further contain, according to the purpose of use, components ordinarily employed for hair cosmetic compositions. Examples include polymer compounds such as cationic cellulose, hydroxylated cellulose and highly polymerized polyethylene oxide; nonionic surfactants such as polyoxyethylene alkyl ethers, polyoxyethylene sorbitan fatty acid esters, glycerin fatty acid esters, polyglycerin fatty acid esters, polyoxyethylene hydrogenated castor oils, sucrose fatty acid esters, polyglycerin alkyl ethers, fatty acid alkanolamides, and alkyl glycosides; anti-dandruffs such as zinc pyrithione and benzalkonium chloride; vitamin preparations; bactericides; anti-inflammatory agents; antiseptics; chelating agents; humectants such as panthenol; colorants such as dyes and pigments; extracts such as extract of Eucalyptus in a polar solvent, protein available from a pearl or a shell having a pear layer or hydrolysate of the protein, protein available from silk or hydrolysate of the protein, protein-containing extract available from seeds of legume plants, Panax ginseng extract, rice bran extract, fucoid extract, camellia extract, aloe extract, Alpinia Leaf extract and chlorella extract; pearl powder such as mica titanium; perfumes; coloring matters; ultraviolet absorbers; antioxidants; and other components described in ENCYCLOPEDIA OF SHAMPOO INGREDIENTS (MICELLE PRESS).

The aqueous hair cosmetic composition of the present invention preferably has a pH (at 25° C.) of from 1 to 5.5 when diluted to 20 times the weight with water. When the pH falls within the above-described range, the resulting composition has a function of recovering the hair damage caused by coloring or the like and giving good flexibility and a supple touch to the hair from wetting to even after drying. The pH is adjusted preferably to from 2.0 to 5.0, more preferably from 2.5 to 4.5 in view of the recovering effect of damaged hair. For pH adjustment, an acid substance such as inorganic acid or organic acid and a basic substance such as sodium hydroxide can be used in combination. Examples of the inorganic acid and organic acid include the above-described ones used for the neutralization of the ether amine as Component (A).

The aqueous hair cosmetic composition of the present invention is prepared by dissolving Components (A) to (C) and other optional components in a solvent containing water and, if necessary, ethanol, 2-propanol, glycerin, propylene glycol or the like. It is provided as an aqueous hair cosmetic composition which is applied before or after shampooing and is rinsed off with water after use, for example, hair rinse, hair conditioner, hair treatment or the like.

### EXAMPLES

The pH of each of the aqueous hair cosmetic compositions which will be described below is measured at 25° C. when the composition is diluted to 20 times its weight with water.

### Examples 1 to 17 and Comparative Examples 1 to 9

Aqueous hair cosmetic compositions having the composition as shown in Tables 1 to 3 were prepared and their “flexibility”, “smoothness”, “moisturized feel”, and “suppleness” were evaluated by the below-described method and criteria. In addition, a fatigue resistance test was carried out.

### Evaluation Method

A bundle of 20 g (from about 15 to 20 cm) of Japanese female hair subjected to cosmetic treatment such as cold waving or bleaching was shampooed. To the resulting hair bundle was applied uniformly 2 g of an aqueous hair cosmetic composition, followed by rinsing with running water for 30 seconds. After towel drying and blow drying, “flexibility”, “smoothness”, “moisturized feel” and “suppleness” of the hair bundle was organoleptically evaluated by a panel of five experts. The total of their scores is shown.

### Evaluation Criteria:

### “Flexibility”

4: Very flexible
3: Flexible
2: Not so flexible
1: Not flexible

### “Smoothness”

4: Very smooth

3: Smooth

2: Not so smooth

1: Not smooth

### “Moisturized Feel”

4: Very moisturized

3: Moisturized

2: Not so moisturized

1: Not moisturized

### “Suppleness”

4: Very supple

3: Supple

2: Not so supple

1: Not supple

### “Fatigue Resistance Test”

In accordance with the method as described in Y. K. Kamath, S. B. Hornby, H. D. Weigmann and S. Ruetsch, J. Cos. Sci., 50, 198-200 (1999), a load of 50 g/hair was applied in repetition and the number of repetitions until the hair was broken was counted. Based on the resulting data, a parameter (characteristic life) of fatigue resistance was calculated as described below.

To statistically analyze the fatigue breakage phenomenon, “Weibull distribution” by which characteristics in variation can be evaluated over a wide range is employed. In the equation (a) of Weibull distribution, a parameter 0 (characteristic life) is determined from the equation (b) available by taking natural logarithm on both sides twice. An approximate curve is determined by plotting each data with an x-axis as ln x and y axis as ln ln {1/[1−F(x)]} and the parameter 0 is calculated from an intercept=b ln θ and gradient=b (equation (c)).

F(x)=1−exp[−(x/θ)^{b}] (a)

ln ln {1/[1−F(x)]}=b ln x−b ln θ (b)

θ(Characteristic life)=exp(−intercept/gradient) (c)

x: the number of repetitions at which the hair is broken

F(x): breakage order/the population

b: shape parameter

θ: characteristic life (number of repetitions at which 63.2% of the populations is broken)

TABLE 1

| | | Examples | | | | | | | | Comparative Examples | | |
|---|---|---|---|---|---|---|---|---|---|---|---|---|
| (Wt.%) | | 1 | 2 | 3 | 4 | 5 | 6 | 7 | 8 | 1 | 2 | 3 |
| (A) | N,N-Dimethyl-3- | 1.0 | 1.0 | 1.0 | 1.0 | 1.0 | 1.0 | 1.0 | 1.0 | — | 1.0 | 1.0 |
| | octadecyloxypropylamine | | | | | | | | | | | |
| (A)′ | Stearyl trimethylammonium | — | — | — | — | — | — | — | — | 1.0 | — | — |
| | chloride | | | | | | | | | | | |
| (B) | Benzyl alcohol | 0.5 | 0.5 | 0.5 | 0.5 | — | 0.5 | 0.5 | 0.5 | 0.5 | — | 0.5 |
| | Benzyloxyethanol | — | — | — | 0.5 | 0.5 | — | — | — | — | — | — |
| (C) | 18-Methyleicosanoic acid | 0.5 | 0.5 | 0.5 | 0.5 | 0.5 | — | — | — | 0.5 | 0.5 | — |
| | 16-Methylheptadecanoic acid | — | — | — | — | — | 0.5 | — | — | — | — | — |
| | 16-Methyloctadecanoic acid | — | — | — | — | — | — | 0.5 | — | — | — | — |
| | Lanolin fatty acid ^{*1} | — | — | — | — | — | — | — | 0.5 | — | — | — |
| (C)′ | Oleic acid | — | — | — | — | — | — | — | — | — | — | 1.0 |
| (D) | Amphipathic amide lipid A ^{*2} | — | 0.5 | — | — | — | — | — | — | — | — | — |
| | Amphipathic amide lipid B ^{*3} | 0.5 | — | — | — | — | — | — | — | — | — | — |
| Others | Stearyl alcohol | 3.0 | 3.0 | 3.0 | 3.0 | 3.0 | 3.0 | 3.0 | 3.0 | 3.0 | 3.0 | 3.0 |
| | Dimethicone ^{*4} | 2.0 | 2.0 | 2.0 | 2.0 | 2.0 | 2.0 | 2.0 | 2.0 | 2.0 | 2.0 | 2.0 |
| | pH Regulator | q.s. | q.s. | q.s. | q.s. | q.s. | q.s. | q.s. | q.s. | q.s. | q.s. | q.s. |
| | (Sodium hydroxide, lactic acid) | | | | | | | | | | | |
| | Ion exchange water | Balance | Balance | Balance | Balance | Balance | Balance | Balance | Balance | Balance | Balance | Balance |
| pH | | 3.2 | 3.2 | 3.2 | 3.2 | 3.2 | 3.2 | 3.2 | 3.2 | 3.2 | 3.2 | 3.2 |
| Evalu- | Flexibility | 20 | 20 | 19 | 18 | 17 | 20 | 18 | 19 | 7 | 10 | 10 |
| ation | Smoothness | 20 | 20 | 20 | 19 | 19 | 18 | 17 | 18 | 6 | 9 | 9 |
| | Moisturized Feel | 19 | 20 | 17 | 16 | 19 | 19 | 19 | 17 | 8 | 8 | 6 |
| | Suppleness | 20 | 20 | 18 | 19 | 19 | 17 | 18 | 17 | 10 | 11 | 5 |
| | Characteristic life θ (times) | 67000 | 69000 | 65000 | 64000 | 62000 | 60000 | 61000 | 62000 | 60000 | 50000 | 37000 |

^{*1} “Crodacid 18-MEA” (Croda Japan K.K.)
^{*2} Amphipathic amide lipid A

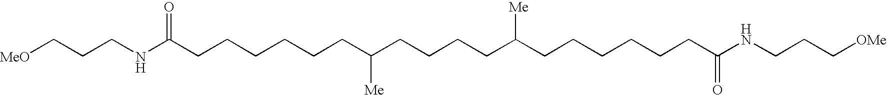

^{*3} Amphipathic amide lipid B

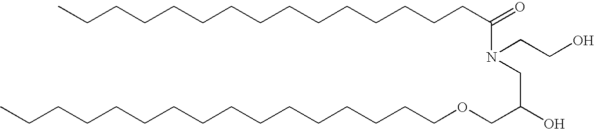

^{*4} “SH200C” (viscosity: 100000 mm²/s, product of Dow Corning Toray)

TABLE 2

| | | Examples | | | | | | | | Comparative Examples | | |
|---|---|---|---|---|---|---|---|---|---|---|---|---|
| (Wt. %) | | 9 | 10 | 11 | 12 | 13 | 14 | 15 | 16 | 4 | 5 | 6 |
| (A) | Stearic acid dimethyl- | 1.0 | 1.0 | 1.0 | 1.0 | 1.0 | 1.0 | 1.0 | 1.0 | — | 1.0 | 1.0 |
| | aminopropylamide | | | | | | | | | | | |
| (A)′ | Stearyl trimethylammonium | — | — | — | — | — | — | — | — | 1.0 | — | — |
| | chloride | | | | | | | | | | | |
| (B) | Benzyl alcohol | 0.5 | 0.5 | 0.5 | 0.5 | — | 0.5 | 0.5 | 0.5 | 0.5 | — | 0.5 |
| | Benzyloxyethanol | — | — | — | 0.5 | 0.5 | — | — | — | — | — | — |
| (C) | 18-Methyleicosanoic acid | 0.5 | 0.5 | 0.5 | 0.5 | 0.5 | — | — | — | 0.5 | 0.5 | — |
| | 16-Methylheptadecanoic acid | — | — | — | — | — | 0.5 | — | — | — | — | — |
| | 16-Methyloctadecanoic acid | — | — | — | — | — | — | 0.5 | — | — | — | — |
| | Lanolin fatty acid*¹ | — | — | — | — | — | — | — | 0.5 | — | — | — |
| (C)′ | Oleic acid | — | — | — | — | — | — | — | — | — | — | 1.0 |
| (D) | Amphipathic amide lipid A*² | — | 0.5 | — | — | — | — | — | — | — | — | — |
| | Amphipathic amide lipid B*³ | 0.5 | — | — | — | — | — | — | — | — | — | — |
| Others | Stearyl alcohol | 3.0 | 3.0 | 3.0 | 3.0 | 3.0 | 3.0 | 3.0 | 3.0 | 3.0 | 3.0 | 3.0 |
| | Dimethicone*⁴ | 2.0 | 2.0 | 2.0 | 2.0 | 2.0 | 2.0 | 2.0 | 2.0 | 2.0 | 2.0 | 2.0 |
| | Lactic acid | 1.5 | 1.5 | 1.5 | 1.5 | 1.5 | 1.5 | 1.5 | 1.5 | 1.5 | 1.5 | 1.5 |
| | pH Regulator | q.s. | q.s. | q.s. | q.s. | q.s. | q.s. | q.s. | q.s. | q.s. | q.s. | q.s. |
| | (Sodium hydroxide, lactic acid) | | | | | | | | | | | |
| | Ion exchange water | Balance | Balance | Balance | Balance | Balance | Balance | Balance | Balance | Balance | Balance | Balance |
| pH | | 3.2 | 3.2 | 3.2 | 3.2 | 3.2 | 3.2 | 3.2 | 3.2 | 3.2 | 3.2 | 3.2 |
| Evaluation | Flexibility | 19 | 20 | 19 | 20 | 18 | 19 | 19 | 18 | 5 | 9 | 9 |
| | Smoothness | 20 | 19 | 19 | 20 | 19 | 20 | 19 | 19 | 7 | 9 | 9 |
| | Moisturized Feel | 19 | 19 | 19 | 20 | 17 | 16 | 17 | 17 | 9 | 9 | 7 |
| | Suppleness | 20 | 20 | 18 | 19 | 18 | 17 | 16 | 17 | 9 | 10 | 7 |
| | Characteristic life θ (times) | 68000 | 65000 | 67000 | 65000 | 63000 | 60000 | 61000 | 60000 | 61000 | 50000 | 35000 |

TABLE 3

| | | Examples | | | | | | | | Comparative Examples | | |
|---|---|---|---|---|---|---|---|---|---|---|---|---|
| (Wt. %) | | 17 | 18 | 19 | 20 | 21 | 22 | 23 | 24 | 7 | 8 | 9 |
| (A) | Octadecyloxy(2-hydroxy- | 1.0 | 1.0 | 1.0 | 1.0 | 1.0 | 1.0 | 1.0 | 1.0 | — | 1.0 | 1.0 |
| | propyl)dimethylamine | | | | | | | | | | | |
| (A)′ | Stearyl trimethyl-ammonium | — | — | — | — | — | — | — | — | 1.0 | — | — |
| | chloride | | | | | | | | | | | |
| (B) | Benzyl alcohol | 0.5 | 0.5 | 0.5 | 0.5 | — | 0.5 | 0.5 | 0.5 | 0.5 | — | 0.5 |
| | Benzyloxyethanol | — | — | — | 0.5 | 0.5 | — | — | — | — | — | — |
| (C) | 18-Methyleicosanoic acid | 0.5 | 0.5 | 0.5 | 0.5 | 0.5 | — | — | — | 0.5 | 0.5 | — |
| | 16-Methylheptadecanoic acid | — | — | — | — | — | 0.5 | — | — | — | — | — |
| | 16-Methyloctadecanoic acid | — | — | — | — | — | — | 0.5 | — | — | — | — |
| | Lanolin fatty acid*¹ | — | — | — | — | — | — | — | 0.5 | — | — | — |
| (C)′ | Oleic acid | — | — | — | — | — | — | — | — | — | — | 1.0 |
| (D) | Amphipathic amide lipid A*² | — | 0.5 | — | — | — | — | — | — | — | — | — |
| | Amphipathic amide lipid B*³ | 0.5 | — | — | — | — | — | — | — | — | — | — |
| Others | Stearyl alcohol | 3.0 | 3.0 | 3.0 | 3.0 | 3.0 | 3.0 | 3.0 | 3.0 | 3.0 | 3.0 | 3.0 |
| | Dimethicone*⁴ | 2.0 | 2.0 | 2.0 | 2.0 | 2.0 | 2.0 | 2.0 | 2.0 | 2.0 | 2.0 | 2.0 |
| | pH Regulator | q.s. | q.s. | q.s. | q.s. | q.s. | q.s. | q.s. | q.s. | q.s. | q.s. | q.s. |
| | (Sodium hydroxide, lactic acid) | | | | | | | | | | | |
| | Ion exchange water | Balance | Balance | Balance | Balance | Balance | Balance | Balance | Balance | Balance | Balance | Balance |
| pH | | 3.2 | 3.2 | 3.2 | 3.2 | 3.2 | 3.2 | 3.2 | 3.2 | 3.2 | 3.2 | 3.2 |
| Evaluation | Flexibility | 20 | 19 | 19 | 18 | 19 | 20 | 18 | 19 | 7 | 9 | 9 |
| | Smoothness | 20 | 19 | 20 | 18 | 18 | 19 | 17 | 18 | 6 | 8 | 10 |
| | Moisturized Feel | 18 | 19 | 18 | 16 | 18 | 18 | 19 | 17 | 8 | 9 | 5 |
| | Suppleness | 19 | 19 | 19 | 18 | 19 | 18 | 17 | 18 | 10 | 10 | 5 |
| | Characteristic life θ (times) | 65000 | 68000 | 64000 | 63000 | 65000 | 62000 | 60000 | 61000 | 60000 | 51000 | 35000 |

### Example 25

### Hair Conditioner (pH 3.3)

| | (wt. %) |
|---|---|
| N,N-Dimethyl-3-octadecyloxypropylamine | 2.0 |
| Stearyl alcohol | 5.0 |
| Dipropylene glycol | 1.0 |
| Benzyl alcohol | 0.5 |
| Phenoxyethanol | 0.1 |
| Glycerin | 5.0 |
| Polypropylene glycol | 2.5 |
| 18-Methyleicosanoic acid | 0.5 |
| Amphipathic amide lipid B | 0.1 |
| Sunflower oil | 0.5 |
| Methylpolysiloxane mixed solution | 2.5 |
| Lactic acid | 1.5 |
| Perfume | 0.4 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 26

### Hair Conditioner (pH 3.3)

| | (wt. %) |
|---|---|
| N,N-Dimethyl-3-octadecyloxypropylamine | 0.5 |
| Stearic acid dimethylaminopropylamide | 2.0 |
| Stearyl alcohol | 5.0 |
| Dipropylene glycol | 1.0 |
| Benzyl alcohol | 0.5 |
| Phenoxyethanol | 0.1 |
| Glycerin | 5.0 |
| Polypropylene glycol | 2.5 |
| 18-Methyleicosanoic acid | 0.5 |
| Amphipathic amide lipid B | 0.1 |
| Sunflower oil | 0.5 |
| Methylpolysiloxane mixed solution | 2.5 |
| Lactic acid | 1.5 |
| Perfume | 0.4 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 27

### Hair Conditioner (pH 3.3)

| | (wt. %) |
|---|---|
| N,N-Dimethyl-3-octadecyloxypropylamine | 0.5 |
| N-Stearyl poly(4)oxyethylene-N,N,N- | 2.0 |
| trimethylammonium chloride | |
| Stearyl alcohol | 5.0 |
| Dipropylene glycol | 1.0 |
| Benzyl alcohol | 0.5 |
| Phenoxyethanol | 0.1 |
| Glycerin | 5.0 |
| Polypropylene glycol | 2.5 |
| 18-Methyleicosanoic acid | 0.5 |
| Amphipathic amide lipid B | 0.1 |
| Sunflower oil | 0.5 |
| Methylpolysiloxane mixed solution | 2.5 |
| Lactic acid | 1.5 |
| Perfume | 0.4 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 28

### Hair Treatment (pH 3.3)

| | (wt. %) |
|---|---|
| N,N-Dimethyl-3-octadecyloxypropylamine | 3.4 |
| Stearyl alcohol | 9.0 |
| Glutamic acid | 1.5 |
| Benzyloxyethanol | 1.0 |
| Dipropylene glycol | 2.0 |
| Phenoxyethanol | 0.1 |
| Amphipathic amide lipid B | 0.1 |
| Dipentaerythritol fatty acid ester | 0.2 |
| 18-Methyleicosanoic acid | 0.5 |
| Methylpolysiloxane mixed solution | 2.5 |
| Highly polymerized methylpolysiloxane• | 2.5 |
| decamethylcyclopentasiloxane mixture | |
| Aminoethylaminopropylsiloxane•dimethylsiloxane | 0.5 |
| copolymer emulsion | |
| Hydroxyethyl cellulose | 0.3 |
| Paraffin | 0.5 |
| Perfume | 0.3 |
| Sodium hydroxide | q.s. |
| Ion exchange water | Balance |

The above-described hair treatment can repair or prevent hair damage and fatigue breakdown and can impart good flexibility and a supple touch to the hair during from wetting to even after drying.

### Example 29

### Hair Conditioner (pH 4.5)

| | (wt. %) |
|---|---|
| Stearic acid dimethylaminopropylamide | 0.5 |
| Stearyl alcohol | 3.0 |
| Benzyloxyethanol | 0.3 |
| Stearyltrimethylammonium chloride | 1.0 |
| Myristic acid | 0.2 |
| Isotridecyl myristate | 0.5 |
| Aminoethylaminopropylsiloxane•dimethylsiloxane | 0.5 |
| copolymer emulsion | |
| Dimethylpolysiloxane | 3.0 |
| Propylene glycol | 1.0 |
| Malic acid | 0.1 |
| 18-Methyleicosanoic acid | 0.5 |
| Amphipathic amide lipid B | 0.1 |
| Perfume | 0.4 |
| Methylparaben | 0.3 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 30

### Hair Treatment (pH 4.0)

| | (wt. %) |
|---|---|
| Behenic acid dimethylaminopropylamide | 2.0 |
| Behenyltrimethylammonium chloride | 0.3 |
| Stearyl alcohol | 4.5 |
| Behenyl alcohol | 1.5 |
| Isononyl isononanoate | 0.5 |
| Methylpolysiloxane mixed solution | 2.5 |
| Aminoethylaminopropylsiloxane•dimethylsiloxane | 0.5 |
| copolymer emulsion | |
| Glycolic acid | 0.5 |
| Malic acid | 0.1 |
| Dipropylene glycol | 3.0 |
| Benzyl alcohol | 0.3 |
| Amphipathic amide lipid B | 0.1 |
| Arginine | 0.2 |
| 18-Methyleicosanoic acid | 0.5 |
| Pantothenyl ethyl ether | 0.1 |
| Perfume | 0.4 |
| Methylparaben | 0.1 |
| Sodium hydroxide | q.s. |
| Ion exchange water | Balance |

The above-described hair treatment can repair or prevent hair damage and fatigue breakdown and can impart good flexibility and a supple touch to the hair during from wetting to even after drying.

### Example 31

### Hair Conditioner (pH 3.3)

| | (wt. %) |
|---|---|
| Octadecyloxy(2-hydroxypropyl)dimethylamine | 2.0 |
| Stearyl alcohol | 5.0 |
| Dipropylene glycol | 1.0 |
| Benzyl alcohol | 0.5 |
| Phenoxyethanol | 0.1 |
| Glycerin | 5.0 |
| Polypropylene glycol | 2.5 |
| 18-Methyleicosanoic acid | 0.5 |
| Amphipathic amide lipid B | 0.1 |
| Sunflower oil | 0.5 |
| Methylpolysiloxane mixed solution | 2.5 |
| Lactic acid | 1.5 |
| Perfume | 0.4 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 32

### Hair Conditioner (pH 3.3)

| | (wt. %) |
|---|---|
| Octadecyloxy(2-hydroxypropyl)dimethylamine | 0.5 |
| Stearic acid dimethylaminopropylamide | 2.0 |
| Stearyl alcohol | 5.0 |
| Dipropylene glycol | 1.0 |
| Benzyl alcohol | 0.5 |
| Phenoxyethanol | 0.1 |
| Glycerin | 5.0 |
| Polypropylene glycol | 2.5 |
| 18-Methyleicosanoic acid | 0.5 |
| Amphipathic amide lipid B | 0.1 |
| Sunflower oil | 0.5 |
| Methylpolysiloxane mixed solution | 2.5 |
| Lactic acid | 1.5 |
| Perfume | 0.4 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 33

### Hair Conditioner (pH 3.3)

| | (wt. %) |
|---|---|
| Hexadecyloxy(2-hydroxypropyl)dimethylamine | 0.5 |
| N,N,N-Trimethylammonium chloride | 2.0 |
| Stearyl alcohol | 5.0 |
| Dipropylene glycol | 1.0 |
| Benzyl alcohol | 0.5 |
| Phenoxyethanol | 0.1 |
| Glycerin | 5.0 |
| Polypropylene glycol | 2.5 |
| 18-Methyleicosanoic acid | 0.5 |
| Amphipathic amide lipid B | 0.1 |
| Sunflower oil | 0.5 |
| Methylpolysiloxane mixed solution | 2.5 |
| Lactic acid | 1.5 |
| Perfume | 0.4 |
| Sodium hydroxide | q.s. |
| Ion exchange water | Balance |

The above-described conditioner can repair or prevent hair damage and fatigue breakdown and can impart good flexibility and a supple touch to the hair during from wetting to even after drying.

### Example 34

### Hair Treatment (pH 3.3)

| | (wt. %) |
|---|---|
| Octadecyloxy(2-hydroxypropyl)dimethylamine | 3.4 |
| Stearyl alcohol | 9.0 |
| Glutamic acid | 1.5 |
| Benzyloxyethanol | 1.0 |
| Dipropylene glycol | 2.0 |
| Phenoxyethanol | 0.1 |
| Amphipathic amide lipid B | 0.1 |
| Dipentaerythritol fatty acid ester | 0.2 |
| 18-Methyleicosanoic acid | 0.5 |
| Methylpolysiloxane mixed solution | 2.5 |
| Highly polymerized methylpolysiloxane• | 2.5 |
| decamethylcyclopentasiloxane mixture | |
| Aminoethylaminopropylsiloxane•dimethylsiloxane | 0.5 |
| copolymer emulsion | |
| Hydroxyethyl cellulose | 0.3 |
| Paraffin | 0.5 |
| Perfume | 0.3 |
| Sodium hydroxide | q.s. |
| Ion exchange water | Balance |

The above-described hair treatment can repair or prevent hair damage and fatigue breakdown and can impart good flexibility and a supple touch to the hair during from wetting to even after drying.

1. An aqueous hair cosmetic composition comprising the following Components (A) to (C):
(A) a tertiary amine type cationic surfactant,
(B) an aromatic alcohol represented by the following formula (2): 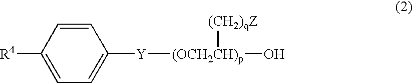
(wherein, R⁴ represents a hydrogen atom, a methyl group or a methoxy group, Y represents a single bond or a linear or branched C₁₋₃ alkylene or alkenylene group, Z represents a hydrogen atom or a hydroxyl group and p and q each stands for a number from 0 to 5),
(C) a branched fatty acid represented by the following formula (3): 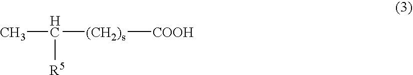
(wherein, R⁵ represents a methyl or ethyl group and s stands for an integer of from 3 to 36) or salt thereof, and water.

2. The aqueous hair cosmetic composition according to claim 1, wherein Component (A) is a tertiary amine compound represented by any one of the following formulas (1-1) to (1-4): 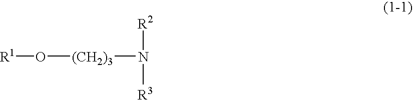
(wherein, R¹ represents a linear or branched C₆₋₂₄ alkyl or alkenyl group, R² and R³ may be the same or different and each represents a C₁₋₆ alkyl group or a group -(AO)ₘH (in which A represents a C₂₋₄ alkylene group, and m stands for a number from 1 to 6 with the proviso that m pieces of A may be the same or different and they may be arranged in any order)),
R—CONH—(CH₂)ᵣ—N(R′)₂ (1-2)
(wherein, R represents an aliphatic C₁₁₋₂₃ hydrocarbon group, R's may be the same or different and each represents a hydrogen atom or a C₁₋₄ alkyl group, and r stands for a number from 2 to 4), and 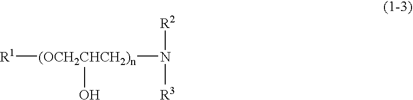
(wherein, R¹, R² and R³ have the same meanings as defined above and n stands for from 1 to 5),
R¹—N(CH₃)₂ (1-4)
(wherein, R¹ has the same meaning as described above), or salt thereof.

3. The aqueous hair cosmetic composition according to claim 1 or 2, wherein a Component (B)/Component (C) weight ratio falls within a range of from 100/1 to 1/10.

4. The aqueous hair cosmetic composition according to any one of claims 1 to 3, further comprising an amphipathic amide lipid as Component (D).
